(19) United States
(12) Patent Application Publication
(10) Pub. No.: US 2008/0015251 A1
(43) Pub. Date: Jan. 17, 2008

(54) Means and method for treating lipotoxicity and other metabolically related phenomena

(75) Inventors: Oren Tirosh, Ness Ziona (IL); Zecharia Madar, Rehovot (IL); Anna Aronis, Petach Tikvah (IL); Erez Ilan, Kibbutz Netzer Sireni (IL)

Correspondence Address: BROWDY AND NEIMARK, P.L.L.C.;624 NINTH STREET, NW, SUITE 300, WASHINGTON, DC, 20001-5303, US

(73) Assignee: Yissum Research Development Co., of The Hebrew University of Jerusalem, Jerusalem (IL)

(21) Appl. No.: 11/783,609

(22) Filed: Apr. 10, 2007

Related U.S. Application Data
(60) Provisional application No. 60/790,543, filed on Apr. 10, 2006.

(51) Int. Cl. A61K 31/341; A61P 3/00; A61P 9/00

(52) U.S. Cl. 514/474

(57) ABSTRACT

A method for treating symptoms of a lipotoxicity-related phenomenon includes introducing into a patient a therapeutically effective synergistic amount of a phenomenon-inhibiting cocktail of a polyphenol (P), thiol antioxidant (To) and ascorbic acid (A) to reduce intracellular reactive oxygen species (ROS) and/or increase intracellular nitrite (NO), so that reduction of such phenomenon is at least 30% greater than the reduction of such phenomenon provided by separate administration of each of P, To and A.

### CROSS REFERENCE

The present patent application claims the priority of US provisional patent application No. 60/790,543 dated 10 Apr. 2006.

A method for treating symptoms of a lipotoxicity-related phenomenon, comprising at least one step of introducing into a patient a therapeutically effective synergistic amount of a phenomenon-inhibiting cocktail, by said introducing, polyphenols (P), thiol antioxidants (To) and ascorbic acid (A) are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient. A cocktail comprising synergistic dosages of polyphenols (P), thiol antioxidants (To) and ascorbic acid (A), wherein the ratios of the components in the cocktail are such that after introducing said cocktail into a patient, a reduction of a lipotoxicity-related phenomenon is at least 30% greater than said phenomenon reduction provided by administration of each of said P, To and A, when separately introduced into said patient.

### FIELD OF THE INVENTION

The present invention generally relates to means and method of synergistically reducing lipotoxicity and other metabolically related phenomena.

### BACKGROUND OF THE INVENTION

The metabolic syndrome comprises a set of metabolic and physiologic risk factors associated with elevated cardiovascular disease risk. The expression of each one of its major factors (hypertriglyceridemia, low high-density lipoprotein cholesterol levels, hypertension, abdominal obesity, and insulin resistance) has been found to be the result of complex interactions between genetic and environmental factors. Moreover, obesity may play a major role in triggering the metabolic syndrome by interacting with genetic variants at candidate genes for dyslipidemia, hypertension, and insulin resistance (Sonnenberg et al; Obes Res. 2004 February; 12(2):180-6).

It has taken nearly 25 years for the concept of a metabolic syndrome to be accepted. Some of the complications of obesity, such as diabetes, hypertension, insulin resistance, and heart disease were more clearly related to the central distribution of fat than to overall level of obesity. This and a variety of other pieces of information led Reaven in 1988 to propose “Syndrome X” called also dysmetabolic syndrome as the name for these findings that clustered with insulin resistance (Bray G A. et al, Champagne Journal of the American Dietetic Association V 104, 1, Jan. 2004, pp 86-89)

Some of the components that can be included in the syndrome are hyperinsulinemia, hypertension, abnormal blood lipids (dyslipidemia), a procoagulant state, vascular abnormalities, inflammatory markers, and hyperuricemia (Kopp Metabolism 52 (2003), 840-844,5., R. D. Brook et al, J Am Coll Nutr 22 (2003), 290-295, A. Onat et al Atherosclerosis 168 (2003), 81-89, A. J. Garber Am Fam Physician 62 (2000), 2633-2642, P. A. Sakkinen et al, Am J Epidemiol 152 (2000), pp. 897-907, R. P. Tracy, Int J Clin Pract Suppl 134 (2003), 10-17, O. Timar, Can J Cardiol 16 (2000), 779-789.)

TABLE 1
Some features and components of metabolic disorder

| Central features | Other components |
|---|---|
| Central adiposity | Microalbuminuria |
| Dyslipidemia including | Procoagulant state including elevated |
| increased plasma | levels of plasminogen activator |
| triglycerides, low plasma | inhibitor-1, von Willebrand factor, |
| HDL, cholesterol, and small | fibrinogen, and factor VII. |
| dense LDL cholesterol | |
| particles | |
| Hypertension | Inflamantory markers including elevated |
| | levels of C-reactive protein (CRP) and |
| | II-6 |
| Hyperglycemia | Vascular abnormalities including |
| | elevated levels of intracellular |
| | adhesion molecule-1, and vascular cell |
| | adhesion molecule |
| Hyperinsulinemia | Insulin resistance |
| Abnormal glucose tolerance | Hyperuricemia |

A great deal of research has been focused on the mechanisms that produce this syndrome. A genetic predisposition clearly underlies susceptibility to the metabolic syndrome (B. L. Wajchenberg, Endocr Rev 21 (2000), pp. 697-738, C. Bouchardet al Endocr Rev 14 (1993), pp. 72-93, H. E. Lebovitz, Int J Clin. Pract Suppl 134 (2003), pp. 18-27. Central adiposity, diabetes mellitus, high-fat diets, aging, medications, physical inactivity, the polycystic ovary syndrome, and low birth weight with imprinting of the brain are other important predisposing factors (M. Yao et al; Int J Obes Relat Metab Disord 27 (2003), pp. 920-932, S. Zhu, Z. Wang et al., Am J Clin Nutr 78 (2003), pp. 228-235, R. Sathyaprakash et al, Curr Diab Rep 2 (2002), pp. 416-422, K. V. Axen, et al. J Nutr 133 (2003), pp. 2244-2249, G. A. Bray and D. H. Ryan, Endocrine 13 (2000), pp. 167-186, S. A. Everson, et al, Diabetes Care 21 (1998), pp. 1637-1643, M. T. Korytkowski, et al, J Clin Endocrinol Metab 80 (1995), pp. 3327-3334, J. Gustat, et al, J Clin Epidemiol 55 (2002), pp. 997-1006,S. Liu et al, Curr Opin Lipidol 12 (2001), pp. 395-404, E. Dahlgren et al., Fertil Steril 61 (1994), pp. 455-460, P. M. Catalano, et al, J Nutr 133 suppl 2 (2003), pp. 1674S-1683S 13-24).

Obesity

That obesity is associated with insulin resistance and type II diabetes mellitus is well accepted. Overloading of white adipose tissue beyond its storage capacity leads to lipid disorders in non-adipose tissues, namely skeletal and cardiac muscles, pancreas, and liver, effects that are often mediated through increased non-esterified fatty acid fluxes. This in turn leads to a tissue-specific disordered insulin response and increased lipid deposition and lipotoxicity, coupled to abnormal plasma metabolic and (or) lipoprotein profiles. Thus, the importance of functional adipocytes is crucial, as highlighted by the disorders seen in both “too much” (obesity) and “too little” (lipodystrophy) white adipose tissue. However, beyond its capacity for fat storage, white adipose tissue is now well recognized as an endocrine tissue producing multiple hormones whose plasma levels are altered in obese, insulin-resistant, and diabetic subjects. The consequence of these hormonal alterations with respect to both glucose and lipid metabolism in insulin target tissues is just beginning to be understood. (Schubert, C. Nature Medicine 10, 322 (2004).

Obesity is accompanied by high plasma levels of nonesterified fatty acids that cause insulin resistance in skeletal muscle and overload the liver with lipid, producing fatty liver and atherogenic dyslipidemia (Scott M. Grundy, JAMA4. 2003;290:3000-3002).

Excess adipose tissue releases increased amounts of plasminogen activator inhibitor 1, contributing to a prothrombotic state. In addition, excess adipose tissue further releases inflammatory cytokines that likely increase levels of C-reactive protein; fat accumulation in the liver moreover may stimulate hepatic cytokine production, which could further increase levels of C-reactive protein. (Diehl A M. Am J Physiol Gastrointest Liver Physiol. 2002; 282:G1-G5).

Diabetes

Type 2 diabetes mellitus is a heterogeneous syndrome characterized by abnormalities in carbohydrate and fat metabolism. The causes of type 2 diabetes are multi-factorial and include both genetic and environmental elements that affect beta-cell function and tissue (muscle, liver, adipose tissue, pancreas) insulin sensitivity (Scheen A J. Acta Clin Belg. 2003 November-December; 58 (6):335-41). Although there is considerable debate as to the relative contributions of beta-cell dysfunction and reduced insulin sensitivity to the pathogenesis of diabetes, it is generally agreed that both these factors play important roles (Faraj M et al, Biochem Cell Biol. 2004 February; 82 (1):170-190. However, the mechanisms controlling the interplay of these two impairments are unclear. A number of factors have been suggested as possibly linking insulin resistance and beta-cell dysfunction in the pathogenesis of type 2 diabetes. A majority of individuals suffering from type 2 diabetes are obese, with central visceral adiposity. Therefore, the adipose tissue should play a crucial role in the pathogenesis of type 2 diabetes. Although the predominant paradigm used to explain this link is the portal/visceral hypothesis giving a key role in elevated non-esterified fatty acid concentrations, two new emerging paradigms are the ectopic fat storage syndrome (deposition of triglycerides in muscle, liver and pancreatic cells) and the adipose tissue as endocrine organ hypothesis (secretion of various adipocytokins, i.e., leptin, TNF-alpha, resistin, adiponectin, implicated in insulin resistance and possibly beta-cell dysfunction). These two paradigms constitute the framework for the study of the interplay between insulin resistance and beta-cell dysfunction in type 2 diabetes as well as between our obesogenic environment and diabetes risk in the next decade; See for example in Pittas A G. et al, Nutr Clin Care. 2003 May-September;6(2):79-88. Clapham J. C. et al, Curr Drug Targets. 2004 April;5(3):309-23.

Coronary Heart Disease

Obesity is a major modifiable CHD risk factor, and the benefits of weight loss are numerous, leading to improvements in several co-morbidities. The global burden of coronary heart disease (CHD) has led to the introduction of international guidelines to minimize the morbidity and mortality that result from this condition (Shirai K., Curr Med Res Opin. 2004 March; 20(3):295-304, Corella D and Ordovas J M., Curr Atheroscler Rep. 2004 May;6(3): 186-96). These guidelines recognize the contribution of multiple risk factors to the development of CHD and advocate a multifaceted approach to treatment. Guidelines advocate lifestyle changes to correct excess bodyweight and improve the CHD risk factor profile; see for example in Semenkovich C F Trends Cardiovasc Med. 2004 February;14(2):72-6nkow J S, Jacobs D R Jr, Steinberger J, Moran A, Sinaiko A R Diabetes Care. 2004 Mar; 27(3):775-80, Fisher M. Heart. 2004 March; 90(3):336-40.

Oxidized LDL (oxLDL) has been shown to play an important role in the pathogenesis of atherosclerosis (Diabetes 53:1068-1073, 2004 Holvoet P Diabetes. 2004 April; 53(4):1068-1073).

In middle-aged people, obesity and dyslipidemia are the strongest predictors of levels of oxLDL. Recently, the association between dyslipidemia and oxidation of LDL has been demonstrated in individuals in the pre-diabetic state. Metabolic syndrome is associated with high risk for atherosclerotic disease, a process thought to involve LDL oxidation.

Hypertension

There is no doubt that obesity is a causative factor in the development of hypertension. Several mechanisms have been proposed; (Scott M. Grundy, M D, JAMA. 2003;290:3000-3002 Ferrannini E, et al. N Engl J Med. 1987;317:350-357), increased sodium retention, activation of the renin-angiotensin system and sympathetic nervous system, intrarenal compression by adipose tissue, and sleep disturbance. Insulin resistance, hyperinsulinemia, or both have been implicated in the pathogenesis of hypertension. Many individuals with hypertension manifest insulin resistance (Reaven, Gerald M. Diabetes Care 27:1011-1012, 2004). The compensatory hyperinsulinemia of insulin resistance has been implicated both in promotion of renal reabsorption of sodium and in overactivity of the sympathetic nervous system (Ibid).

Inflammation

Insulin resistance recently has been linked with a proinflammatory state (elevations of C-reactive protein (Scott M. Grundy, M D, JAMA. 2003;290:3000-3002).

As shown in many studies, a primary insulin resistance without obesity recapitulates many of the metabolic effects of obesity. This finding further emphasizes the connection between metabolic disturbance and inflammatory response.

That elevations of inflammatory markers are associated with metabolic risk factors and with accelerated atherosclerotic disease is well established. What is lacking is an adequate causal explanation for these associations. Undoubtedly, the causal connections are complex, and in many instances, undiscovered. Ridker (Ridker P M. Circulation. 2003; 107:363-369) has demonstrated a connection between inflammatory markers and major coronary events. One well-established risk factor, cigarette smoking, is a known cause of elevations of inflammatory markers (Frohlich M, et al. Eur Heart J. 2003; 24:1365-1372).

The direct atherogenic effect of smoking in promoting arterial wall inflammation appears to be well established. The recognition that obesity, insulin resistance, and the risk factors of the metabolic syndrome are associated with high levels of inflammatory markers provides another causal pathway to atherosclerotic disease. Certainly several of the metabolic risk factors are atherogenic independent of inflammatory markers. Another line of causation is suggested in the current article, namely, that the acute phase reactants are proinflammatory in their own right. The advantages of enhancement of the inflammatory response by acute phase reactants in response to infectious agents are clear. Further exploration of this mechanism seems worthwhile. The possibility that inflammatory markers are a response to products released from arterial inflammation remains an attractive hypothesis. The reduction in these markers in response to drugs that should dampen arterial inflammation appears to support this mechanism (Scott M. Grundy, M D, Inflammation, JAMA. 2003; 290:3000-3002).

Lipotoxicity

Recently, attention has been focused on the excessive accumulation of triglycerides (TG) within the liver as part of this metabolic syndrome (den Boer M, et al, Arterioscler Thromb Vasc Biol. 2004 April; 24(4):644-).

The relentless decline in B-cell function frequently observed in type 2 diabetic patients, despite optimal drug management, has variously been attributed to glucose toxicity and lipotoxicity (Bugianesi E, et al, Dig Liver Dis. 2004 Mar; 36(3):165-73, R. Paul Robertson et al, Diabetes 53:S119-S124, 2004). The former theory posits hyperglycemia, an outcome of the disease, as a secondary force that further damages 13-cells. The latter theory suggests that the often-associated defect of hyperlipidemia is a primary cause of β-cell dysfunction. Evidence has shown that patients with type 2 diabetes continually undergo oxidative stress. Elevated glucose concentrations increase levels of reactive oxygen species in β-cells. Islets have intrinsically low antioxidant enzyme defenses, that antioxidant drugs and overexpression of antioxidant enzymes protect β-cells from glucose toxicity, and that lipotoxicity, to the extent it can be attributable to hyperlipidemia, occurs only in the context of preexisting hyperglycemia, whereas glucose toxicity can occur in the absence of hyperlipidemia.

The mechanisms whereby chronic elevations of glucose and/or lipids might damage β-cells are subjects of intense clinical and laboratory investigation (Fernandez-Checa J C. Ann Hepatol. 2003 April-June; 2(2):69-75). Whether one or the other of these forces is more important or whether they may be interrelated is another important consideration. Chronic oxidative stress is a mechanism whereby glucose excess can damage β-cells and that lipotoxicity requires hyperglycemia to exert harmful effects, whereas glucose toxicity does not require concomitant hyperlipidemia to do so.

It appears that fat accumulation in the liver is associated with several features of insulin resistance even in normal-weight and moderately overweight subjects. Nonetheless, from these observations in humans it remains unclear to what extent hepatic steatosis is a cause rather than a consequence of the metabolic syndrome.

In general, there is continuous cycling and redistribution of non-oxidized fatty acids between different organs. The amount of triacylglycerol (TG) in an intrinsically normal liver is not fixed but can readily be increased by nutritional, metabolic, and endocrine interactions involving TG/free fatty acid partitioning and TG/FFA metabolism. Several lines of evidence indicate that hepatic TG accumulation is also a causative factor involved in hepatic insulin resistance. Complex interactions between endocrine, metabolic, and transcriptional pathways are involved in TG-induced hepatic insulin resistance. Therefore, the liver participates passively and actively in the metabolic derangements of the metabolic syndrome (Bugianesi E, et al, Dig Liver Dis. 2004 March; 36(3):165-73, R. Paul Robertson et al, Diabetes 53:S119-S124, 2004, Fernandez-Checa J C. Ann Hepatol. 2003 April-June; 2(2):69-75).

The TG content of hepatocytes is regulated by the integrated activities of cellular molecules that facilitate hepatic TG uptake, fatty acid synthesis, and esterification on the one hand (“input”) and hepatic fatty acid oxidation and TG export on the other (“output”). Steatosis occurs when “input” exceeds the capacity for “output.” The liver acts in concert with other organs in the orchestration of inter-organ fatty acid/TG partitioning.

Endothelial Dysfunction

The endothelium is a dynamic autocrine/paracrine organ that regulates vascular tone and the interaction of the vessel wall with circulating substances and blood cells. The endothelium produces vasodilators and vasoconstrictors that, under normal physiologic conditions, are in balance. A major vasodilator is nitric oxide (NO), which has multiple vascular-protective actions. These include inhibition of vascular smooth muscle cell (VSMC) growth and migration, platelet aggregation and thrombosis, monocyte adhesion, inflammation, and oxidation (Willa A. Hsueh Manuel J. Quifiones M D The American Journal of Cardiology 92(4) 18 Aug. 2003, pp 10-17). In contrast, vasoconstrictors, such as angiotensin II, promote vascular damage and inflammation. Endothelial dysfunction is an early step in the atherogenic process. Classic and nontraditional risk factors have been shown to be associated with endothelial dysfunction leading to impairment of NO release, increased oxidative stress, and loss of protection against the atherogenic process (Yokoyama M Curr Opin Pharmacol. 2004 April;4(2):110-5). Recent evidence has demonstrated that insulin resistance in the absence of overt type 2 diabetes or the metabolic syndrome results in endothelial dysfunction in peripheral and coronary vasculature. Evidence also indicates that endothelial dysfunction itself could contribute to insulin resistance. Thus, treatment strategies that attenuate cardiovascular disease may also attenuate insulin resistance progression. Elucidating the common mechanisms that mediate these events will be important in understanding their intimate relation. Until the answers to these questions are available, early recognition and treatment of risk factors associated with either insulin resistance or cardiovascular disease are critical in the prevention of atherosclerosis (Higashi Y, Yoshizumi M. Pharmacol Ther. 2004 April; 102(1):87-96).

The presence of endothelial dysfunction early in the spectrum of insulin resistance strongly suggests that vascular damage, potentially associated with oxidation, inflammation, and thrombosis, also is present. Therefore, early recognition and treatment of insulin resistance, at least with lifestyle modification and aggressive management of risk factors, are critical in the prevention of atherosclerosis and potentially in the prevention of diabetes (Kawano H, Ogawa H Curr Drug Targets Cardiovasc Haematol Disord. 2004 March;4(1):23-33). Whether approaches that improve endothelial function in insulin resistance also decrease cardiovascular risk remains to be determined.

In light of the above, it is clear that a means and method for reducing phenomena associated with Syndrome X, Metabolic Syndrome, lipotoxicity and/or hepatic steatosis would fulfill a long felt need.

### SUMMARY OF THE INVENTION

It is an object of the invention to disclose a method for treating symptoms of a lipotoxicity-related phenomenon, comprising at least one step of introducing into a patient a therapeutically effective synergistic amount of a phenomenon-inhibiting cocktail, by said introducing, polyphenols (P), thiol antioxidants (To) and ascorbic acid (A) in synergistic ratios are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient.

It is another object of the invention to disclose the above method wherein said ratios are effective bioactive dosage ratios ranging from 1:1000 to 1000:1; especially from 1:100 to 100:1; and especially from 1:10 to 10:1.

It is another object of the aforementioned invention wherein the polyphenol is resveratrol.

It is another object of the aforementioned invention wherein the thiol antioxidant is N-acetyl cysteine.

It is another object of the aforementioned invention wherein the lipotoxicity-related phenomenon is a metabolic disorder selected from a group consisting of Metabolic syndrome, Syndrome X (SX), lipotoxicity (L), Arterial, Heart and Related Diseases (AHRD), diabetes related disorders, obesity related disorders, coronorary heart disease disorders, Non-Alcoholic Steatohepatitis (NASH), triacylglycerol (TG), Free Fatty Acid (FFA) or any combination thereof.

It is another object of the aforementioned invention wherein the method for treating arterial and related diseases especially relates to AHRD phenomena, selected from the group consisting of hypertension, hyperlipidemia, atherosclerosis, arteriosclerosis, coronary artery disease, myocardial infarction, congestive heart failure, stroke, and angina pectoris.

It is another object of the aforementioned invention wherein the step of introducing in the aforementioned method is provided orally.

It is another object of the aforementioned invention wherein the step of introducing in the aforementioned method is provided intravenously.

It is another object of the aforementioned invention to provide a cocktail comprising synergistic dosages of polyphenols (P), thiol antioxidants (To) and ascorbic acid (A), wherein the ratios of the components in the cocktail are such that after introducing said cocktail into a patient, a reduction of a lipotoxicity-related phenomenon is at least 30% greater than said phenomenon reduction provided by administration of each of said P, To and A, when separately introduced into said patient.

It is another object of the aforementioned invention to provide the cocktail such that the ratios are effective bioactive dosage ratios ranging from 1:1000 to 1000:1; especially from 1:100 to 100: 1; and especially from 1:10 to 10:1.

The cocktail as defined above, wherein said lipotoxicity-related phenomenon is a metabolic disorder selected from a group consisting of Syndrome X (SX), Metabolic syndrome, lipotoxicity (L), Arterial, Heart and Related Diseases (AHRD), Non-Alcoholic Steatohepatitis (NASH), triacylglycerol (TG), diabetes, obesity, corner heart disease, Free Fatty Acid (FFA) or any combination thereof.

It is another objective of the aforementioned invention to provide the cocktail wherein said polyphenol is resveratrol.

It is another objective of the aforementioned invention to provide the cocktail wherein said thiol antioxidant is N-acetyl cysteine.

Lastly, it is another objective of the invention to provide a food additive comprising the cocktail as defined in any of the definitions above.

### BRIEF DESCRIPTION OF THE FIGURES

In order to understand the invention and to see how it may be implemented in practice, a preferred embodiment will now be described, by way of non-limiting example only, with reference to the accompanying figures in which;

FIG. 1. presents LE analysis;

FIG. 2. presents an analysis of reactive oxygen species (ROS) levels and DNA fragmentation;

FIG. 3. illustrates cell treatment with LE facilitates changes in reactive oxygen species (ROS) and reduced glutathione (GSH) levels;

FIG. 4. illustrates that treatment with LE decreases J774.2 macrophage viability and caspase-3 activity;

FIG. 5. presents cell viability analysis following treatment with tert-butyl hydroperoxide (TBH) or LE in J774.2 macrophages;

FIG. 6. illustrates cells treated with proapoptotic cycloheximide (CH) and with lipid emulsion (LE);

FIG. 7. illustrates the protective effect of N-acetylcysteine (NAC);

FIG. 8. illustrates antioxidant protection against triacylglycerol (TG)-induced lipotoxicity;

FIG. 9. illustrates the effect of LE on nitrite production and intracellular ROS levels in rat isolated hepatocytes;

FIG. 10. illustrates iNOS (A) eNOS (B) mRNA levels and iNOS (C) protein expression in rat isolated hepatocytes exposed to LE and NAC; and,

FIG. 11. illustrates Nitrite levels in the culture medium after exposure of rat isolated hepatocytes to LE and NAC, resveratrol, or ascorbate.

### DETAILED DESCRIPTION OF THE EMBODIMENTS

The following description is provided, alongside all chapters of the present invention, so as to enable a person skilled in the art to make use of said invention and sets forth the best modes contemplated by the inventor of carrying out this invention. Various modifications however will remain apparent to those skilled in the art, since the generic principles of the present invention have been defined specifically to provide a means and method for decreasing oxidative stress-mediated lipotoxicity and/or hepatic steatosis in mammals.

The term “CH” refers hereafter to cycloheximide. The term “DMSO” refers hereafter to dimethyl sulfoxide.

The term “FACS” refers hereafter to flow cytometer. The term “FADD” refers hereafter to FAS-associated protein with death domain.

The term “FFA” refers hereafter to free fatty acid.

The term “FID” refers hereafter to flame ionization detector.

The term “GSH” refers hereafter to reduced glutathione.

The term “HPLC” refers hereafter to high-performance liquid chromatography.

The term “GC” refers hereafter to gas chromatography.

The term “LDL” refers hereafter to low-density lipoprotein.

The term “LE” refers hereafter to lipid emulsion.

The term “NAC” refers hereafter to N-acetylcysteine.

The term “NBT” refers hereafter to nitroblue tetrazolium.

The term “PV” refers hereinafter to peroxide value.

The term “PBS” refers herein after to phosphate-buffered saline.

The term “PI” refers hereinafter to propidium iodide.

The term “PUFA” refers hereinafter to polyunsaturated fatty acid.

The term “Rf” refers hereinafter to retention factor.

The term “TBH” refers herein after to tert-butyl hydroperoxide.

The term “TG” refers hereinafter to triacylglycerol.

The term “VLDL” refers hereinafter to very-low-density lipoprotein.

The term “AP-1” refers hereafter to activator protein 1.

The term “BSO” refers hereafter to buthionine sulfoximine.

The term “DTT” refers hereafter to dithiothreitol.

The term “eNOS” refers hereafter to endothelial nitric oxide synthase.

The term “GAPDH” refers hereafter to gylceraldehyde-3-phosphate dehydrogenase.

The term “GSH” refers hereafter to reduced glutathione.

The term “H2DCF-DA” refers hereafter to dichlorodihydrofluorescein diacetate.

The term “iNOS” refers hereafter to inducible nitric oxide synthase.

The term “LE” refers hereafter to lipid emulsion.

The term “NAC” refers hereafter to N-acetyl-L-cysteine.

The term “NASH” refers hereafter to nonalcoholic steatohepatitis.

The term “NF-κB” refers hereafter to nuclear factor icB.

The term “NO” refers hereafter to nitric oxide.

The term “PMSF” refers hereafter to phenylmethylsulfonyl fluoride.

The term “ROS” refers hereafter to reactive oxygen species.

The term “SNAP” refers hereafter to S-nitroso-N-acetyl-penicillamine.

The term “TG” refers hereafter to triacylglycerol.

The term “NF” refers hereafter to tumor necrosis factor.

The term “TPN” refers hereafter to total parenteral nutrition.

The term “lipotoxicity-related phenomenon” refers to any phenomenon associated with a metabolic disorder selected from a group consisting of Syndrome X (SX), Metabolic disorder, lipotoxicity (L), Arterial, Heart and Related Diseases (AHRD), Non-Alcoholic Steatohepatitis (NASH), triacylglycerol (TG), Free Fatty Acid (FFA) or any combination thereof.

The term “reduction”, such as reduction of Syndrome X (SX), refers hereafter to reduction of the referred phenomena (e.g., SX), to below 30% of its occurrence as compared with reduction of said phenomenon provided by administration of each component of the phenomenon—inhibiting cocktail when separately introduced into said patient.

The term “separately introduced”, refers hereafter to individual dosages of therapeutically active components separately administered to a patient at intervals over time.

The term “synergistic amount”, refers hereafter to a therapeutically effective amount of a phenomenon-inhibiting cocktail (TEPIC) such that reduction of said phenomenon is at least 30% greater than said reduction of said phenomenon provided by administration of each of said P, To and A, when separately introduced into said patient or a TEPIC where the individual amounts of P, To and A in the cocktail are at least 30% less than the amounts of individual dosages of therapeutically active components separately administered to a patient at intervals over time to produce the same reduction of referred phenomena.

Elucidation of death pathways in macrophages resulting from exposure to triacylglycerols (TG), mechanisms which may be relevant to the development of atherosclerosis are described below:

Cell Culture

Murine J774.2 macrophages were cultured in RPMI medium enriched with 10% fetal calf serum, 1% glutamine, and 1% penicillin-streptomycin. Cells were maintained in an incubator with temperature (378C) and CO₂ (5%) control. Prior to experimental procedures, macrophages were seeded on 6-well plates at a concentration of 50,000 cells/ml.

Lipid Emulsion (LE) Treatment

Before treating the cells with a soybean oil TG-based lipid emulsion (Lipofundin 10%, Braun, Germany), the hydrolysis level of the FFA was evaluated by thin-layer chromatography (TLC). The LE was analyzed relative to TG and FFA standards on a silica gel plate (Merck, Germany). The running solvent contained petrol ether, diethyl ether, and acetic acid in a volumetric ratio of 80:20:1. The retention factor (Rf) of the compounds on the plates was then visualized with iodine.

The peroxide value (PV) of the commercial emulsion was measured using the American Oil Chemists' Society's method. Briefly, the lipids were extracted from the LE by chloroformic extraction (1:1:2 LE: chloroform:methanol). Extracted oil (5 mg) was dissolved in a 30-mi acetic acid:chloroform (3:2) mixture and 0.5 ml of saturated potassium iodide solution and 30 ml of distilled water were added. The mixture was titrated with 0.01 M sodium thiosulfate until its yellow color almost disappeared.

Then 0.5 ml of a 1% (w/v) starch solution was added to the mixture, and the titration continued until all of the color disappeared. The PV was calculated using the equation: PV (milliequivalents peroxide per kilogram sample)=S×N×1000/g sample, where S=ml Na2S2O3 and N=normality of the Na2S2O3 solution.

LE was added to the cell culture at a concentration of 0.1% (w/v) TG (1 mg lipids/ml). The physiological range of TG in plasma is up to 1.5 mg/ml. The cells were incubated with the LE for 12, 24, or 48 h, and then washed twice with phosphate-buffered saline (PBS), and intracellular ROS and cell viability were measured. Other agents, such as antioxidants or cycloheximide (CH), were added as specified in the relevant figure legends.

Detection of Cellular Fatty Acid Profile with High-Resolution Gas Chromatogragphy Analysis.

The cellular concentrations of fatty acids were measured using gas chromatography (GC). The cells in medium were centrifuged (600 g, 5 min) and resuspended in PBS. Protein concentration was determined by the Bradford method, cells were centrifuged again at 600g and the supernatant was removed. A mixture of chloroform: methanol (2:1) was added to the cells, and samples were mixed and kept at room temperature for 30 min. 1222A (See for example Aronis et al., Free Radical Biology & Medicine 38 (2005) 1221-1230).

Distilled water (20%, v/v) was then added and the phases separated. The chloroform phase was dried under nitrogen. An internal standard of C17-fatty acid was added and the dried samples were resuspended in toluene and treated with a hydrolysis-methylation reagent (MetPREP, Alltech, Ill.) according to the manufacturer's directions. Analysis was performed with an HP-4890 (Agilent Technologies) GC equipped with a flame ionization detector (FID) and DB-WAX capillary column (60 m, 0.32 mm i.d., df=0.25 Am) (J and W Scientific, Folsom, Calif.). The samples were injected at an injector temperature of 260° C. and split 1/80 for 1 min. Operating conditions were as follows: the column was held at 180° C. for 2 min and then increased by 10° C./min to 230° C. where it was held for 20 min. Helium was used as the carrier gas with a linear velocity of 31 cm/s. Detector temperature was 250° C. Quantification of the results was based on the area of the internal standard peak.

Cell Viability

Cell membrane integrity was detected as previously described. Briefly, cells were stained with 2 Ag/ml propidium iodide (PI) and measured by flow cytometry, (FACS Calibur, Becton Dickinson, Calif.) at the following fluorescence setting: excitation at 488 nm and emission at 575 nm. Data were collected from 10,000 cells.

DNA Integrity

Cells exposed to LE were centrifuged (600 g, 5 min). The pellet was resuspended in 1% (w/v) paraformaldehyde, incubated for 30 min, and centrifuged. The resultant pellet was resuspended in a solution containing 50 Ag/ml PI, 0.1% (w/v) sodium citrate, and 0.1% (v/v) Triton X-100. The permeabilized cells were kept in the dark at 48 C for 2 h. DNA integrity was analyzed by FACS, excitation at 488 nm and emission at 575 nm. Data were collected from 10,000 cells.

ROS Measurements

Intracellular ROS were detected using a H2DCF-DA fluorescent probe. The cells were incubated with 25 AMH2DCF-DA for 30 min at room temperature. The fluorescence was measured in a FACS, with excitation at 488 nm and emission at 530 nm. Data were collected from 10,000 cells.

Superoxide was measured by the use of nitroblue tetrazolium (NBT). After 12, 24, and 48 h exposure to LE, the medium was removed, and fresh medium containing 0.1% (w/v) NBT was added to the cells. Incubation took place in an incubator with controlled humidity (378C, 5% CO2) for 15 min. The cells were then centrifuged (600 g, 5 min), the supernatant was removed, and the pellet was treated with 1 ml dimethyl sulfoxide.

(DMSO) to extract the formazan. After centrifugation, absorbance of the supernatant was measured by spectrophotometer at 520 nm. The results were adjusted to milligram protein. Annexin V-PI double staining Cells were washed twice in phosphate-free binding buffer (10 mM Hepes, 140 mM NaCl, and 2.5 mM CaCl2,pH 7.4) and centrifuged, and the supernatant was removed. Annexin V was dissolved in the same buffer (0.2 Ag/ml) and added to the cells. After a 30-min incubation at room temperature in the dark, 0.2 Ag/ml PI was added, and measurements were made by FACS, at settings FL1(excitation 488 nm, emission 530 nm) and FL3 (excitation 488 nm, emission 675 nm).

Determination of Caspase-3 (DEV Dase) Activity

Necrotic cell death is a major pathway in the absence of caspase activation. Classical apoptotic stimuli such as Fas activation are reported to facilitate the necrotic pathways in cells that do not express caspase-8 and do not activate downstream caspases in response to oligomerization of Fas-associated protein with death domain (FADD). For analysis of caspase activity, cells were incubated for 2 h in ice-cold PBS containing 0.5% Triton X-100 and 5 mM dithiothreitol (DTT). The suspension of permeabilized cells was agitated slightly and centrifuged at 14,000 g. The clear supernatant was tested for caspase-3 activity as previously described, using the caspase-3 substrate Ac-DEVD-AMC (Calbiochem, Darmstadt, Germany). Incubation was conducted in 200 Al of reaction mixture containing approximatelyl mg sample protein, in the dark, at 308 C for 4 h (linear phase of the reaction). Caspase-3 activity was expressed as arbitrary fluorescence units (AU) per mg. protein.

Measurement of Glutathione

Reduced glutathione (GSH) was measured by highpressure liquid chromatography (HPLC). The cells and GSH standard were dissolved in 4% (v/v) metaphosphoric acid and run in running buffer (50 mM KH2PO4 and 2% v/v acetonitrile, pH 2.7) using a Synergy 4 A Polar-RP 80A column (Phenomenex, Torrance, Calif.) when the cell potential was 800 mV. Detection was made by electrochemical detector. The results were reported as nanomole per milligram protein.

Statistical Analysis

Data were analyzed by ANOVA. Differences were considered significant at probability levels of P b 0.05.

The groups were compared using Fisher's exact test.

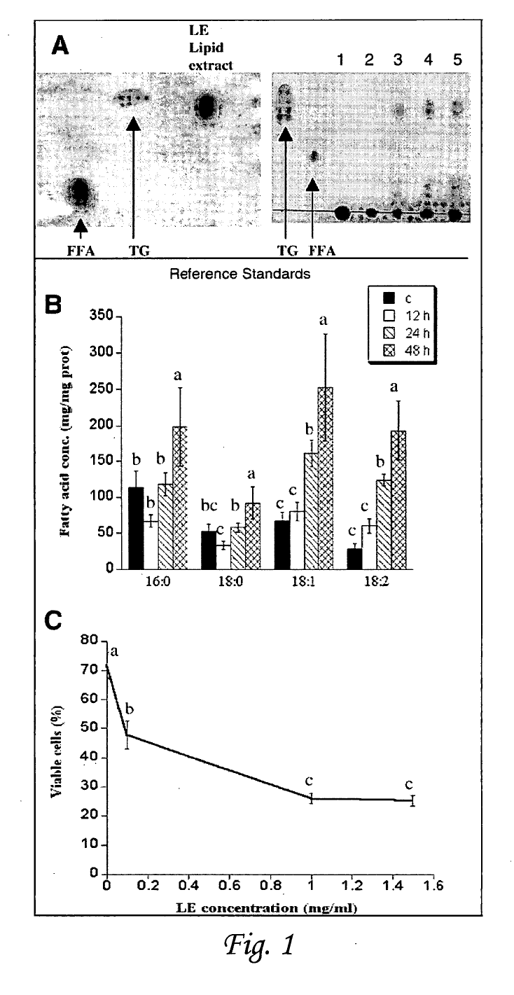

Reference is now made to FIG. 1, presenting LE analysis, cellular uptake of fatty acids, and dose-dependent lipotoxicity of macrophages exposed to LE (A, left).

Lipids were extracted from LE and subjected to TLC. The retention factor (Rf) was compared to free fatty acid (FFA) and triacylglycerol (TG) standards. No significant amount of FFA was seen in the emulsion.

Analysis of decomposition of TG to FFA in the cell culture medium (FIG. 1A, right); cell culture medium (1), cell culture medium collected from cells after 48 h (2), cell culture medium supplemented with LE collected from cells after 12 h (3), same as 3 but collected after 24 h (4), same as 3 but collected after 48 h (5);

J774.2 macrophages were incubated with 1 mg/ml LE (0.1%) for 12, 24, and 48 h, and the fatty-acid profile was determined by gas chromatography (FIG. 1B). Control represents untreated cells seeded and kept in culture for 48 h. Bars (amounts of Ag fatty acid/mg protein) represent averages of three experiments F SD. Different letters indicate statistical differences (P b 0.05) between the cellular concentrations of each fatty acid analyzed at different times of exposure to LE.

J774.2 macrophages were incubated with LE (0.01, 0.1, and 0.15%) for 48 h (FIG. 1C). Cell viability was then measured. Each point on the curve represents an average of three experiments F SD. Different letters indicate statistical differences, P b 0.05.

FIG. 1 underlines a novel aspect of the invention, and discloses on a cellular level a method according another embodiment of the preset invention, useful for treating symptoms of a lipotoxicity-related phenomenon, wherein said phenomenon is intra cellular accumulation of triacylglycerol (TG) and/or Free Fatty Acid (FFA), comprising at least one step of introducing into a patient a therapeutically effective synergistic amount of a phenomenon-inhibiting cocktail, by said introducing, polyphenols (P), thiol antioxidants (To) and ascorbic acid (A) are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient. The ratios are effective bioactive dosage ratios ranging from 1:1000 to 1000:1; especially from 1:100 to 100:1; and especially from 1:10 to 10:1.

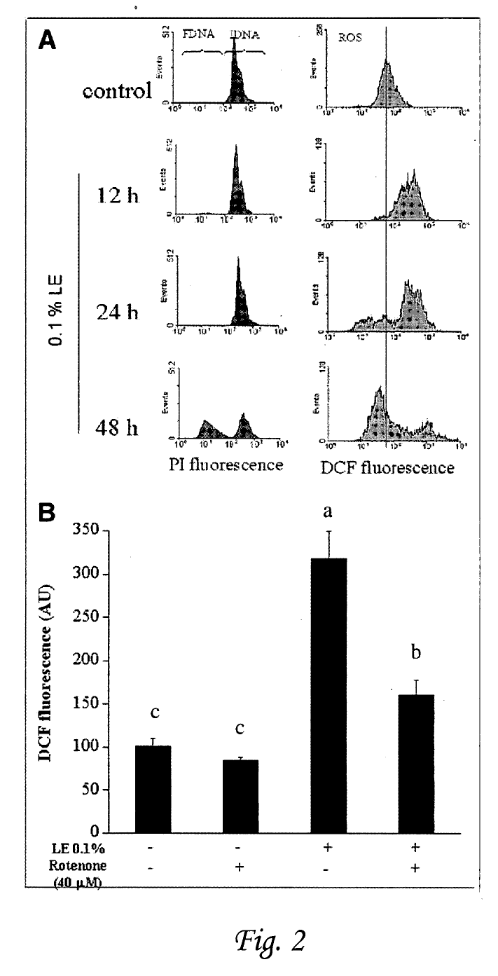

Reference is now made to FIG. 2, disclosing an analysis of reactive oxygen species (ROS) levels and DNA fragmentation in J774.2 cells following exposure to LE. J774.2 macrophages were incubated with 0.1% TG LE for 12, 24, and 48 h. Control represents untreated cells seeded and kept in culture for 48 h. DNA fragmentation analysis (FIG. 2A, left histogram column) and ROS levels (FIG. 2A, right histogram column) were measured by flow cytometer, as described under Materials and methods. Each histogram is representative of three experiments. J774.2 macrophages were incubated with 0.1% TGm LE for 24 h (FIG. 2B). Rotenone (40 AM) was added to the control and LE-treated cells 5 min prior to adding H₂DCF-DA. ROS levels were measured by flow cytometer, as described under Materials and methods. Bars represent averages F SD of three experiments. Different letters indicate statistical differences, P b 0.05.

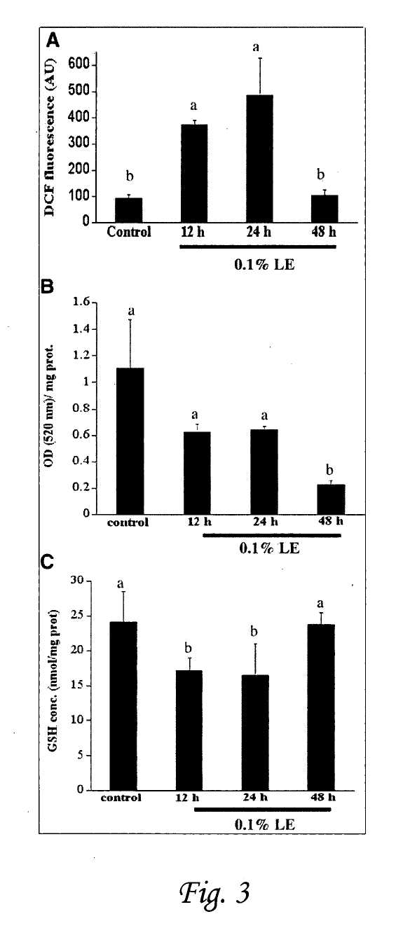

Reference is now made to FIG. 3, illustrating cell treatment with lipid emulsion (LE) facilitates changes in reactive oxygen species (ROS) and reduced glutathione (GSH) levels.

J774.2 macrophages were incubated with 0.1% LE for 12, 24, and 48 h. Control represents untreated cells seeded and kept in culture for 48 h. (A) Levels of H₂DCF-reacting ROS, (B) superoxide levels, and (C) GSH concentrations were measured as described under Materials and methods. Bars represent averages F SD of three experiments. Different letters indicate statistical differences, P b 0.05.

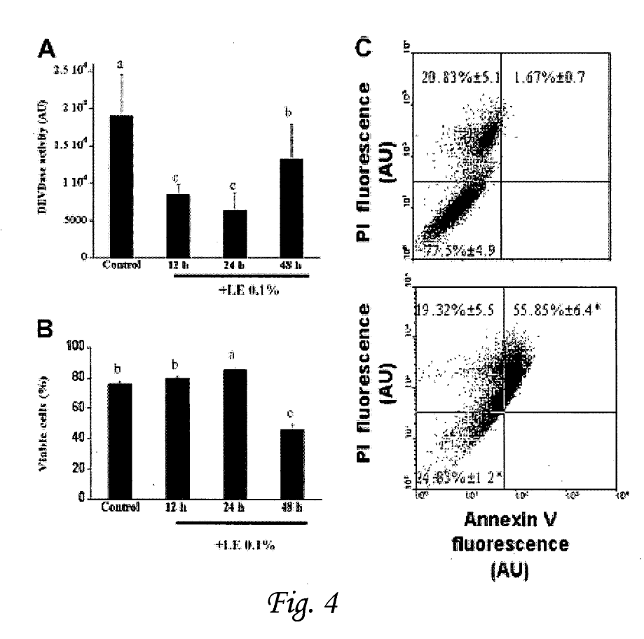

Reference is now made to FIG. 4, illustrating that treatment with lipid emulsion (LE) decreases J774.2 macrophage viability and caspase-3 activity.

Cells were incubated with LE for 12, 24, and 48 h. Control represents untreated cells seeded and kept in culture for 48 h.

Caspase-3 activity as measured by DEVDase activity (FIG. 4A).

Cell viability measured by exclusion of PI (membrane integrity). Bars represent means F SD of three experiments. Different letters indicate statistical differences, P b 0.05 (FIG. 4B).

Necrosis vs apoptosis assay: dual staining of cells with propidium iodide (PI) and Annexin V was performed (FIG. 4C). Fluorescence was measured by a flow cytometer as described under Materials and methods. Upper plot represents the control cells and lower plot represents cells treated with LE for 48 h. In each plot: lower left quadrant represents viable cells; upper left, partial loss of membrane integrity (uptake of PI but not Annexin V); lower right, apoptotic cells; and upper right, necrotic cells. Each quadrant carries information about the percentage of cells framed in it from an average of three experiments F SD; * statistical significance from the upper plot, P b 0.05.

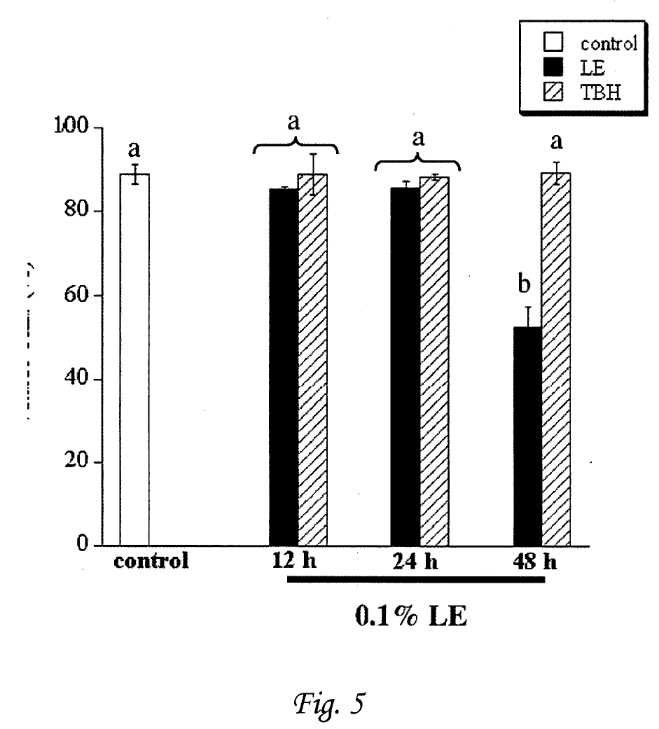

Reference is now made to FIG. 5, presenting cell viability analysis following treatment with tert-butyl hydroperoxide (TBH) or LE in J774.2 macrophages.

After determining a peroxide value of 12 mmol/kg TG, the J774.2 macrophages were exposed to an equimolar concentration of TBH (12 AM) for 12, 24, and 48 h, and its effect on cell viability was compared to the effect of exposure to LE. Control represents untreated cells seeded and kept in culture for 48 h. Bars represent mean F SD of three experiments. Different letters indicate statistical difference, P b 0.05.

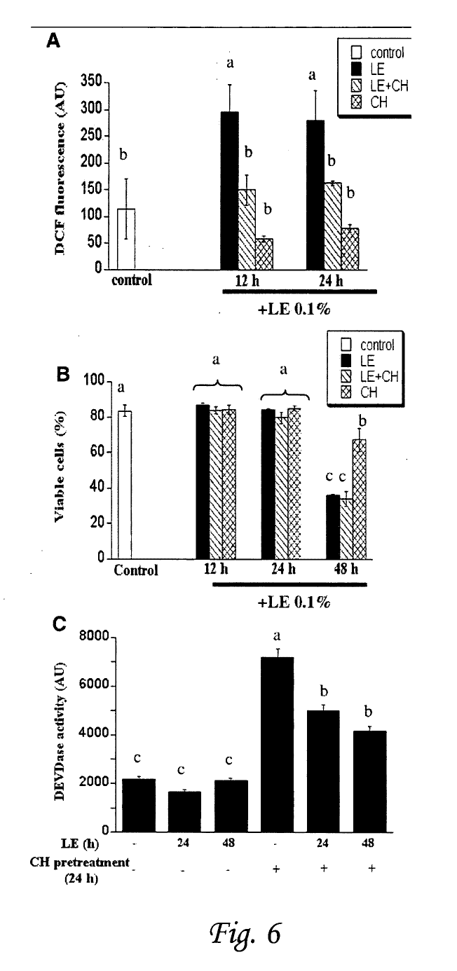

Reference is now made to FIG. 6, illustrating cells treated with proapoptotic cycloheximide (CH) to activate caspase and with lipid emulsion (LE): reactive oxygen species (ROS) production, caspase-3 activity, and cell-death parameters were evaluated. Control represents untreated cells seeded and kept in culture for 24 h for A and 48 h for B and C. (A) ROS measured following 0.1% LE, 0.1 AM CH, and combined treatment. (B) Cell viability measured in LE, CH, and combined treatment. (C) Cells were treated with CH for 24 h and then exposed to LE for the next 24 and 48 h. Caspase-3 activity was measured as described under Materials and methods. Bars represent averages F SD of three experiments. Different letters indicate statistical differences, P b 0.05.

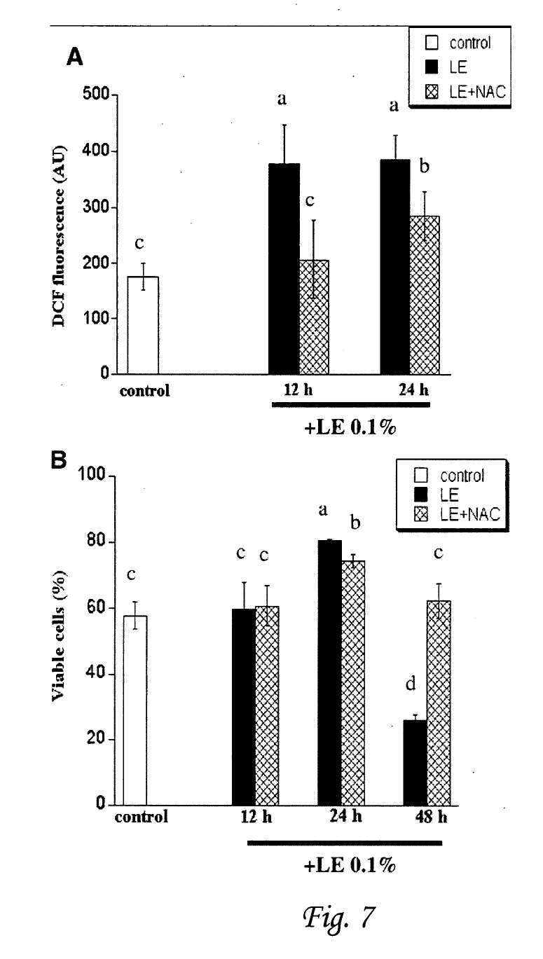

Reference is now made to FIG. 7 illustrating the protective effect of N-acetylcysteine (NAC). NAC plays a protective role in triacylglycerol (TG)-induced lipotoxicity. Cells were treated with 0.1% LE in the presence or absence of NAC (0.5 mM) for 12 or 24 h. Control represents untreated cells seeded and kept in culture for 24 h for A and 48 h for B. Measurements of reactive oxygen species (ROS) (A) and cell viability (B) revealed the protective role of NAC against increased intracellular ROS production and cellular death. n=3, different symbols indicate statistical differences, P b 0.05.

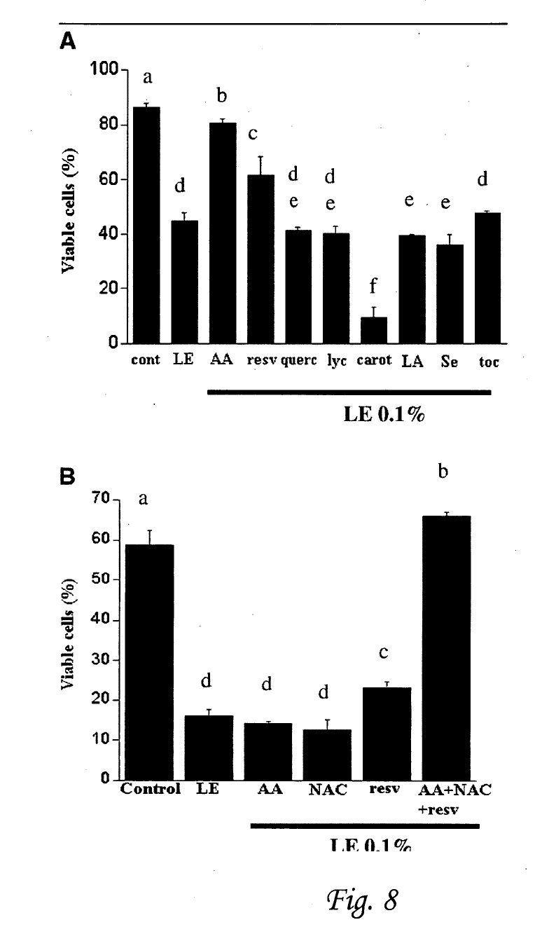

FIG. 7 presents cellular level evidence of the protective effect of NAC, and hence indicates a method according to another embodiment of the present invention. This method is especially adapted for treating symptoms of a lipotoxicity-related phenomenon, namely the increase of intracellular reactive oxygen species (ROS) and decrease of NO when cells are exposed to LE, comprising at least one step of introducing into a patient a therapeutically effective synergistic amount (see viable cell evidence in FIGS. 8A and 8B below) of a phenomenon-inhibiting cocktail, by said introducing thiol antioxidant is N-acetyl cysteine (P), thiol antioxidants (To) and ascorbic acid (A) are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient. The ratios are effective bioactive dosage ratios ranging from 1:1000 to 1000:1; especially from 1:100 to 100:1; and especially from 1:10 to 10:1.

FIGS. 8A and 8B. Antioxidant protection against triacylglycerol (TG)-induced lipotoxicity.

Control represents untreated cells seeded and kept in culture for 48 h. (A) Cells were treated with 0.1% lipid emulsion (LE) in the presence orabsence of the following antioxidant compounds: ascorbic acid (AA, 0.5 mM), resveratrol (resv, 0.2 mM), quercetin (querc, 0.2 mM), lycopene (lyc, 10 AM), h-carotene (carot, 10 AM), a-tocopherol (toc, 0.2 mM), lipoic acid(LA, 0.25 mM), and selenium (Se, 1 AM). (B) Cells were treated with 0.1% LE in the presence or absence of: ascorbic acid (50 AM), NAC (50 AM), or resveratrol (20 AM), or their combination. n=3, different symbols indicate statistical differences, P b 0.05.

Reference is now made to FIG. 8, illustrating antioxidant protection against triacylglycerol (TG)-induced lipotoxicity. FIG. 8 clearly demonstrates on a cellular level the core rationale of the invention, and discloses a method according one embodiment of the preset invention, useful for treating symptoms of a lipotoxicity-related phenomenon, namely TG-related metabolic disorder, comprising at least one step of introducing into a patient a therapeutically effective synergistic amount of a phenomenon-inhibiting cocktail, by said introducing, polyphenols (P), thiol antioxidants (To) and ascorbic acid (A) are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient. The ratios are effective bioactive dosage ratios ranging from 1:1000 to 1000:1; especially from 1:100 to 100:1; and especially from 1:10 to 10:1.

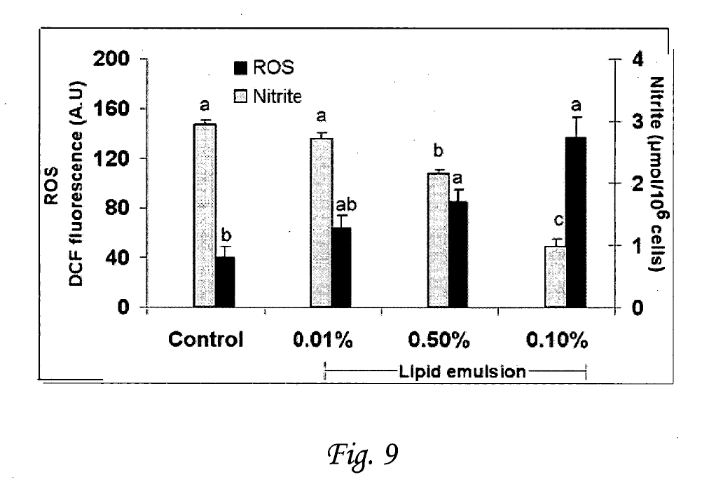

FIG. 9 presents the effect of LE on nitrite production and intracellular ROS levels in rat isolated hepatocytes. Values are means SE, n=6. Means without a common letter differ, P=0.05.

FIG. 9 substantially reinforces, again, on a cellular level another core rationale of the invention, and discloses a method according another embodiment of the preset invention, useful for treating symptoms of a lipotoxicity-related phenomenon, namely the increase of intracellular reactive oxygen species (ROS) and decrease of NO when cells are exposed to LE, comprising at least one step of introducing into a patient a therapeutically effective synergistic amount of a phenomenon-inhibiting cocktail, by said introducing, polyphenols (P), thiol antioxidants (To) and ascorbic acid (A) are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient. The ratios are effective bioactive dosage ratios ranging from 1:1000 to 1000:1; especially from 1:100 to 100:1; and especially from 1:10 to 10:1.

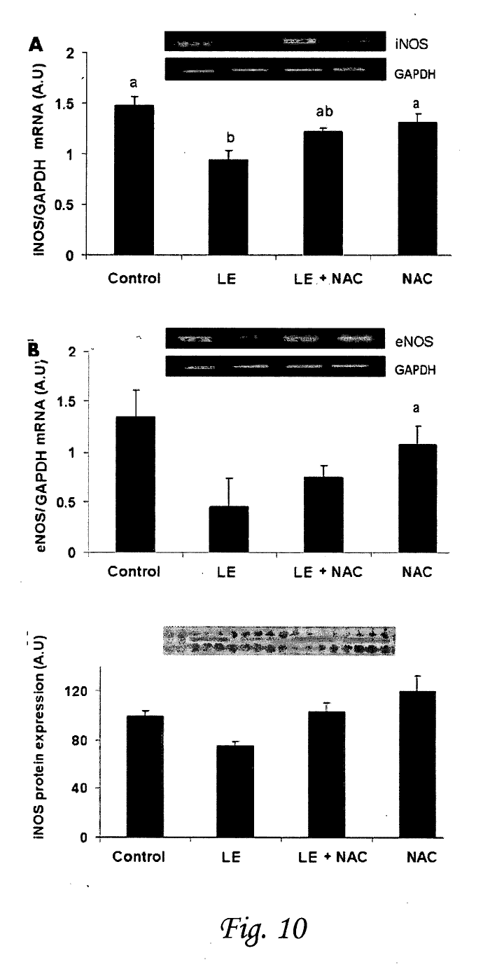

FIG. 10 illustrates iNOS (A) eNOS (B) mRNA levels and iNOS (C) protein expression in rat isolated hepatocytes exposed to LE (0.1%) and NAC(3 mmol/L) for 48 h. Values are means SE, n=6, Means without a common letter differ, P=0.05. ANOVA (iNOS mRNA): LE, P=0.0013;NAC, NS; LE xNAC, P=0.0128. ANOVA (eNOS mRNA): LE, P=0.0188; NAC, NS; LE×NAC, NS. ANOVA (iNOS Protein): LE, P=0.0184; NAC, P=0.0069; LE×NAC, NS.

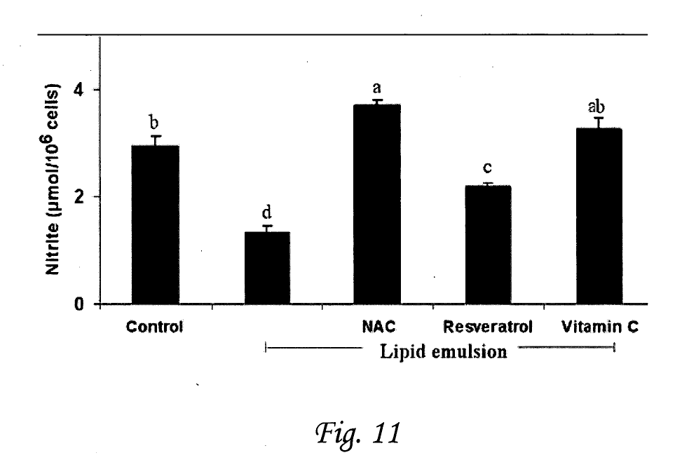

FIG. 11 presents Nitrite levels in the culture medium after exposure of rat isolated hepatocytes to LE (0.1%) and NAC (3 mmol/L), resveratrol (600 mol/L), or ascorbate (3 mmol/L). Values are means SE, n=6. Means without a common letter differ, P=0.01.

FIG. 11 teaches another core rationale of the invention, and discloses on a cellular level a method according to another embodiment of the present invention, useful for treating symptoms of a lipotoxicity-related phenomenon, namely the increase of intracellular reactive oxygen species (ROS) and decrease of NO when cells are exposed to LE, comprising at least one step of introducing into a patient a therapeutically effective synergistic amount of a phenomenon-inhibiting cocktail, by said introducing, polyphenols (P), thiol antioxidants (To) and ascorbic acid (A) are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient. The ratios are effective bioactive dosage ratios ranging from 1:1000 to 1000:1; especially from 1:100 to 100:1; and especially from 1:10 to 10:1.

Hence, the results illustrated in the present invention establishes the method according yet another embodiment of the preset invention, useful for treating symptoms of a lipotoxicity-related phenomenon, wherein these phenomena are selected from a group consisting of Syndrome X (SX), lipotoxicity (L), Arterial, Heart and Related Diseases (AHRD), diabetes related disorders, obesity related disorders, coronary heart disease Non-Alcoholic Steatohepatitis (NASH), triacylglycerol (TG), Free Fatty Acid (FFA) or any combination thereof, comprising at least one step of introducing into a patient a therapeutically effective synergistic amount of a phenomenon-inhibiting cocktail, by said introducing, polyphenols (P), thiol antioxidants (To) and ascorbic acid (A) are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient. The ratios are effective bioactive dosage ratios ranging from 1:1000 to 1000:1; especially from 1:100 to 100:1; and especially from 1:10 to 10:1.

Results

Cellular change in fatty acid profile (lipid uptake) and cytotoxicity, see for example A. Aronis et al., Free Radical Biology & Medicine 38 (2005) 1221-1230.

TLC analysis of LE revealed undetectable amounts of FFA in the commercial LE (FIG. 1A left). We analyzed the hydrolysis of TG during the experiment. Cells in culture were exposed to TG at a concentration of 1 mg lipid/ml for 12, 24, and 48 h. After that TLC analysis showed the presence of TG in the cell culture medium; almost no FFA were present (FIG. 1A right). Following this evaluation, macrophages were treated with 1 mg/ml LE used as a source for TG.

Cellular lipid composition was measured at 12, 24, and 48 h after LE treatment and in untreated cells (FIG. 1B). Cellular levels of 18:1 and 18:2 fatty acids increased over time. This correlates well with the LE composition, as soybean is rich in oleic and linoleic acids. This result indicates that unsaturated fatty acids from the LE were incorporated into the cells. After determining that the TG treatment results in intracellular lipid accumulation, we sought to evaluate lipotoxicity in the macrophage cell line. A dose-dependent increase in cell death was observed in macrophages treated with elevated concentrations of LE (FIG. 1C), indicating TG's lipotoxic effect.

Cellular ROS Production and Glutathione Level

To study the effect of TG on cellular redox status, the level of cellular ROS was measured with the fluorescent marker H2DCF-DA (FIG. 2A, right). FACS analysis showed that a 12-h exposure to 1 mg/ml TG enhances ROS production in the cell population. However, after 24-h exposure to TG, two cell populations (low and high ROS) could be distinguished. Most of the cells exhibited enhanced ROS levels, but a secondary population of cells had lower ROS levels than in controls. Cells with low ROS levels became dominant at 48 h. In order to interrupt the steady-state production of ROS and to identify the source of the TG-induced ROS, the cells were treated with rotenone, a mitochondrial complex I inhibitor. Rotenone was added to the LE-treated cells 5 min before the H2DCF-DA probe used for the ROS measurement.

Inhibition of complex 1 significantly decreased ROS production in TG-treated cells (FIG. 2B). The data indicate that ROS were generated by an endogenous cellular source and are not the result of oxidized lipids delivered from the cell-culture medium. Short-term (15 min) treatment with FCCP to uncouple the mitochondria and to decrease leakage of ROS from the electron-transfer chain due to more efficient transferring of electrons to oxygen also decreased cellular ROS production but not as effectively as rotenone (data not shown).

Comparative analysis of DNA status indicated 50% DNA fragmentation 48 h after TG treatment. Therefore, ROS function is an early signal, coming prior to activation of the endonucleases that degrade cellular DNA. DNA degradation occurred only as a late event together with loss of membrane integrity (FIG. 2A, left; FIG. 4B). In an NBT reduction assay, no increase in superoxide production was observed after exposure of the cells to LE for 12, 24, and 48 h (FIG. 3B). Moreover, 48 h of exposure culminated with a significant decrease in superoxide levels. Changes in the cellular antioxidant status occurred, reflecting oxidative stress. Measurements of GSH showed consumption of this antioxidant in the presence of a high level of ROS (FIGS. 3A and C).

Caspase-3 Activity and Type of Cellular Death

Treatment with LE did not activate caspase-3 activity, as measured with DEVDase (FIG. 4A). Surprisingly, basal caspase-3 activity was suppressed by exposure to the LE. Measurement of cell viability revealed that in 24 h, FIG. 3. Cell treatment with lipid emulsion (LE) facilitates changes in reactive oxygen species (ROS) and reduced glutathione (GSH) levels. J774.2 macrophages were incubated with 0.1% LE for 12, 24, and 48 h. Control represents untreated cells seeded and kept in culture for 48 h. (A) Levels of H2DCF-reacting ROS, (B) superoxide levels, and (C) GSH concentrations were measured as described under Materials and methods. Bars represent averages F SD of three experiments. Different letters indicate statistical differences, P b 0.05. A. Aronis et al. /Free Radical Biology & Medicine 38 (2005) 1221-1230 1225. 1226 A. Aronis et al. /Free Radical Biology & Medicine 38 (2005)1221-1230

TG treatment led to a small, but significant increase in viable cells. These data indicate that for the first 24 h, the apoptotic pathway was suppressed in the macrophages, most likely via a high level of ROS, which are known as suppressors of caspase activity. Longer exposure, for 48 h, resulted in cell death at the rate of 50% (FIG. 4B). Cellular staining with Annexin V and PI indicated activation of the necrotic pathway (FIG. 4C).

TG treatment resulted in a loss of membrane integrity to both PI and Annexin V (upper right quadrant of the plot), indicating necrotic cell death (FIG. 4C).

Tert-butyl hydroperoxide (TBH) treatment and cell viability PV values were measured to evaluate lipid peroxidation levels in LE, and a PV of 12 mmol/kg LE was found. To confirm that lipotoxicity was not the result of such levels of lipid hydroperoxides, cells were treated with TBH at the equimolar concentration of 12 AM (FIG. 5). No loss in viability was observed after 48 h of exposure to TBH (FIG. 5). In comparison, exposure to LE for 48 h caused a significant decrease in cell viability.

Effect of TG on Apoptotic Cells

To further understand the effect of TG on caspase-3 activity, the proapoptotic protein-synthesis inhibitor CH was used. CH is known to activate cellular signaling, resulting in caspase activation and apoptosis. Pretreatment for 24 h with CH led to increased caspase-3 activity (FIG. 6A). CH treatment alone decreased ROS production in the cells, allowing caspase-3 activity in the higher reducing environment. TG treatment following CH resulted in cell death after 48 h, indicating that protein-synthesis inhibition does not prevent the cell-death effect of TG (FIG. 6B). However, TG treatment following CH significantly elevated the cellular ROS levels and partially suppressed the caspase-3 activation (FIG. 6C). Therefore, a higher oxidation state in lipotoxicity suppresses caspase-3 activity and intrinsic apoptosis capacity and may lead to necrotic cell death.

Protective Effect of Antioxidants

The capacity of antioxidants to prevent lipotoxicity was evaluated. A thiol compound, N-acetylcysteine (NAC), was used to prevent ROS production and cell death (FIG. 7). TG induced ROS production was suppressed by treatment with 0.5 mM NAC (FIG. 7A). NAC also protected the cells against TG-induced cell death (FIG. 7B).

A series of additional antioxidants were screened for their ability to prevent lipotoxicity (FIG. 8A). Ascorbic acid (0.5 mM) and resveratrol (0.2 mM) significantly decreased the rate of cell death. Other water- and lipid-soluble antioxidants, such as quercetin, lycopene, h-carotene, atocopherol, selenium, and racemic lipoic acid, did not have any protective effect or even enhanced the rate of cell death. When the protective antioxidants NAC, ascorbic acid, and resveratrol were examined together at one-tenth their concentrations used in the previous experiment (more closely approximating bioavailable levels), a synergistic protective effect was observed (FIG. 8B). These combined compounds afforded full protection against TG-induced lipotoxicity.

Evaluation the effects of triacylglycerol (TG) on nitric oxide (NO) production, expression of endothelial (e) and inducible (i) nitric oxide synthase (NOS) and variables related to oxidative stress.

Collagenase was purchased from Worthington Biochemical. Recombinant tumor necrosis factor (TNF)-α, TriReagent was purchased from Sigma, and Reddymix™ was obtained from ABgene. Lipid emulsion containing 63.8% saturated short- and medium-chain fatty acids [(6:0 to 12:0), 4% 16:0; 1.6% 18:0; 8.5% 18:1; 19.5% 18:2; 2% 18:3; 0.6% 20:4 20:5 22:6] (17) and vitamin E (200 mg/L; Lipofundin 20%) was a gift from Uri Kogan (Luxembourg Pharmaceuticals, Israel). All cell culture materials were purchased from Biological Industries and all other chemicals were purchased from Sigma.

Hepatocyte Isolation

Rat hepatocytes were isolated as described by Berry et al. (18). The cells were suspended at a concentration of 2 □109 cells/L in DMEM containing 10% fetal calf serum, 100 mg/L penicillin, 100 mg/L streptomycin, and 100 mg/L gentamicin and plated onto 10-cm plates. The cultures were incubated at 37° C. and used 3-4 h after plating. All experimental procedures using rats were approved by the Institutional Animal Care Committee of the Hebrew University of Jerusalem.

Cell Culturing and Treatment.

Isolated hepatocytes were seeded at 2 □109 cells/L; after 4 h, the culture medium was replaced and cells were incubated for 48 h with 0.01-0.1% LE. Rotenone, S nitroso-N-acetyl-penicillamine (SNAP), buthionine sulfoximine (BSO), anti oxidants, or TNF-α were added to the culture medium in specific experiments. At the end of the experiment, the medium was collected and the cells were harvested for further assays.

TLC for Determination of FFAs.

TG and FFAs were separated using TLC on DC-Plastikfolien 60, thickness 0.2 mm (Merck). TLC plates were loaded with standards of TG and FFAs along with medium samples and placed in a solvent system comprised of petroleum ether:diethyl ether:acetic acid (80:19:1, by vol) for 50-60 min (19). Visualization was performed by iodine staining.

Determination of Cellular Fatty Acid Content.

The concentrations of FFAs were measured using GC (15). Briefly, cell lipid content was extracted with a mixture of chloroform:methanol (2:1). After the addition of C17 internal standard (0.1 mg for 106 cells) and 20 □L hydrolysis-methylation reagent (MetPREP) to each extract, the samples were suspended in 50 □L toluene. The samples were injected and analyzed by GC using a flame ionization detector.

Protein Expression (Western Blot Analysis).

Cells were scraped and lysed in 750 μΛ of lysis buffer (20 mmol/L Tris, pH 7.8; 0.1% Nonidet P-40; 100 mmol/L NaCl; 50 mmol/L NaF; 10% glycerol, 1 mmol/L sodium orthovanadate). Lysates were centrifuged at 8500×g for 10 min. The supernatant was collected and used for the analysis of eNOS and iNOS.

For the determination of p65 and p-c-Jun, nuclear extracts were prepared by suspending the cells in hypotonic buffer [20 mmol/L HEPES, 10 mmol/L KCl, 1 mmol/L MgCl2, 0.5 mmol/L dithiothreitol (DTT), 0.1% Triton X 100, 20% glycerol, 2 mmol/L phenylmethylsulfonyl fluoride (PMSF), and protease inhibitor cocktail] followed by centrifugation at 1600 g for 10 min. The supernatant was discarded. The pellets were suspended in hypertonic buffer (20 mmol/L HEPES, 10 mmol/L KCl, 1 mmol/L MgCl2, 0.5 mmol/L DTT, 0.1% Triton X 100, 20% glycerol, 2 mmol/L PMSF, 420 mmol/L NaCl, and protease inhibitor cocktail) and incubated on a shaker for 4 min at 4° C. Subsequently, the samples were centrifuged at 35,000 g for 10 min and the supernatant was collected.

Protein concentration was determined by the Bradford method (20) using bovine serum albumin as a standard. Samples were boiled for 5 min with SDS sample buffer; 60 μg of protein per sample was loaded onto a 10% SDS-polyacrylamide gel. Electroblots were blocked in Tris buffer NaCl-Tween (TBST) containing 5% skim milk powder at room temperature. Western blot analysis with a specific antibody against iNOS (Biomol), eNOS, p65, and p-c-Jun antibodies (Santa Cruz Biotechnology) was carried out. The antibodies were diluted in TBST buffer+5% skim milk and left overnight at 4° C. After a TBST washing procedure, the blots were incubated with horseradish-peroxidase labeled anti-rabbit antibody (Pierce) for 1 h at room temperature. The immune reaction was detected by enhanced chemiluminescence. Bands were quantified by scanning densitometry and expressed as arbitrary units.

Determination of Nitrite Concentrations in Culture Media (Griess Reaction).

Nitrite in culture media was measured by the Griess reaction (21). The values obtained were compared with standards of sodium nitrite dissolved in the cell culture media. Nitrite release was calculated and expressed in μmol/106 cells.

Total RNA Isolation and Reverse Transcription PCR Analysis.

Total RNA was prepared using TriReagent. Analyses of mRNA levels of iNOS, eNOS, and gylceraldehyde-3-phosphate dehydrogenase (GAPDH) were performed using RT-PCR. The synthesis of cDNA was carried out using a Reverse-it (ABgene) first-strand synthesis kit. The reaction was conducted at 40-55° C. for 50 min. Finally, the reaction was terminated by incubation at 70° C. for 15 min. PCR amplification was performed in the Biometra T personal cycler instrument (Biometra). Cycles were performed at 94° C. for 30 s followed by 57° C. for 30 s and 72° C. for 30 s. cDNA was incubating with ReddyMix and gene-specific PCR primers, designed using Primer 3 software (Whitehead Institute for Biomedical Research).

The primers were synthesized by MBC: eNOS, forward primer, 5′-GAGCATACCCGCACTTCTGT-3′, and reverse primer, 5′-GAAGATATCTCGGGCAGCAG-3′; INOS, forward primer, 5′CAGCACAGAGGGCTCAAAGG-3′, and reverse primer, 5′-TCGTCGGCCAGCTCTTTCT-3′. As a loading control, RNA was hybridized with a probe of the housekeeping gene, GAPDH, forward primer, 5′-TCCGCCCCTTCCGCTGATG-3′, and reverse primer, 5′-CACGGAAGGCCATGCCAGTGA-3′.

PCR products were electrophoresed on 0.1% agarose gel containing 5 □L ethidium bromide, and the gel image was quantified using Doc-it gel image analysis program (UVP). The number of cycles for all the genes was selected within the linear part of a standard curve.

Reduced Glutathione (GSH) Measurement.

GSH was measured using HPLC (15). Hepatocytes were suspended in 4% metaphosphoric acid and analyzed in running buffer (50 mmol/L KH₂PO₄ and 2% acetonitrile, pH 2.7) in a Synergy 4-μm Polar-RP 80A column (Phenomenex) when the cell potential was 800 mV. Detection was made by an electrochemical detector. The results were adjusted to protein levels of the samples.

Cell Viability TG Accumulation, and ROS.

Cell membrane integrity (15), TG accumulation (22), and intracellular ROS levels (23, 24) were detected by flow cytometer (FACScalibur BD). Hepatocytes were stained with 2 mg/L DNA-interchelating dye propidium iodide, which is excluded by viable cells; with 1 mg/L Nile red, which accumulates in intracellular lipid droplets; or with 50 mmol/L dichlorodihydrofluorescein diacetate (H2DCF-DA), a probe that has high reactivity to hydrogen peroxide, lipid hydroperoxide, and hydroxyl radicals and low reactivity to superoxide anions (23,24). Fluorescence settings were as follows: excitation at 488 nm and emission at 575 nm for propidium iodide and Nile red and 488 nm and recorded at 530 nm for H2DCF-DA. Data were collected from 10,000 cells.

Statistical Analysis.

The significance of the differences between means was determined by Student's t test when a single comparison was performed. When multiple comparisons were carried out, the significance was tested using either 1- or 2-way ANOVA, depending on the number of factors considered. When necessary, data were log transformed to achieve stabilized variance. A post-hoc test (Tukey-Kramer) was performed when the interaction between treatments was significant. Differences were considered significant at P=0.05. JMP version 3.1.6 (SAS Institute) was used for all analyses.

Triacylglycerol inhibits NO systems in hepatocytes 2091. Lipid accumulation in rat isolated hepatocytes. Hepatocyte uptake of TG after 48 h of incubation with 0.1% LE was examined. The hepatocytes' lipid content increased by 40% (Table 1) following 48 h incubation with LE.

TABLE 2
Fatty acid concentrations in rat isolated hepatocytes
(10⁶ cells) incubated with or without 0.1% LE for 48 h¹

| Fatty acid | Control | LE, mol/10⁶ cells |
|---|---|---|
| 16:0 | 24 ± 0.3 | 31 ± 1.6* |
| 16:1 | 1.3 ± 0.4 | 1.7 ± 0.2 |
| 18:0 | 21 ± 0.8 | 26.2 ± 1.5* |
| 18:1 | 7.6 ± 0.2 1 | 6.3 ± 1.0* |
| 18:2 | 20 ± 0.7 | 30 ± 2.0* |
| 18:3 | 1.6 ± 0.6 | 1.6 ± 0.5 |

¹Values are means; SEM, n _ 4;
*Different from control, P = 0.05.

The composition as well as the total fatty acid content was altered after incubation with the LE. The uptake of the fatty acids 16:0, 18:0, 18:1, and 18:2 was significantly increased after incubation with LE (Table 1).

Increases in Nile red fluorescence in the hepatocytes between 0 and 48 h of exposure to LE indicated a time-dependent increase in hepatocyte TG content

TABLE 3
Uptake of TG in rat isolated hepatocytes exposed to 0.1%
LE for 0-48 h¹, 2 Time TG content (Nile red fluorescence)³

| h | AU |
|---|---|
| 0 | 60 ± 3.3^{c} |
| 12 | 86 ± 5.8^{b} |
| 24 | 97 ± 8.1^{b} |
| 48 | 137 ± 9.6^{a} |

¹Values are means; SEM, n _ 4. Means in a column without a common superscript letter differ, P = 0.05.
²Data were log transformed to achieve stabilized variance.
³AU, arbitrary units.

The recovery of TG from the culture medium after 48 h of incubation was 86% as determined by TLC. FFAs were not detected in the culture medium using TLC.

Effect of LE on nitrite and ROS levels in rat isolated hepatocytes. Nitrite levels in the culture medium were measured after exposure to increased levels of LE. LE dose dependently decreased the synthesis of nitrites levels (r □0.99, P □0.006; FIG. 1).

Nitrite levels were decreased 67% by exposure to 0.1% LE. After exposure to the LE, ROS levels were increased 250% (FIG. 1) and were inversely correlated with nitrite levels (r=0.98, P=0.0085; FIG. 1). Cell viability was measured to determine whether the reduction in the nitrite levels was a result of cell death or regulation of NO synthesis. Viability of approximately 90% was measured in control cultures collected after 12, 24, and 48 h of incubation. LE at concentrations ranging from 0.01 to 0.1% did not cause cell death (P=0.1; data not shown) LE-treated cells exhibited a 37% reduction in iNOS mRNA expression (FIG. 2A), whereas eNOS mRNA expression was reduced 67% (FIG. 2B). The reduction of iNOS and eNOS mRNA was accompanied by a concomitant decrease in iNOS protein levels (FIG. 2C) and a marked trend toward reduction of eNOS (data not shown) protein levels (P=0.07). SNAP (NO donor) added to the culture medium for 48 h had no effect on ROS levels in the presence or absence of LE (Table 3).

TABLE 4
Intracellular ROS levels in rat isolated hepatocytes
incubated with or without 0.1% LE and SNAP for
48 h1,² Treatment ROS (DCF fluorescence)3

| | AU |
|---|---|
| Control 100 | 6 |
| LE 253 | 24 |
| SNAP 133 | 8 |
| SNAP _ LE 285 | 15 |
| 2-way ANOVA, | P-values |
| LE | P < 0.0001 |
| SNAP | P = 0.049 |
| SNAP × LE | NS |

¹Values are means, SEM, n _ 6^{;}
²Data were log transformed to achieve stabilized variance^{;}
³AU, arbitrary units; NS, not significant.

Incubation of cells with rotenone increased intracellular ROS levels (Table 4).

TABLE 5
Intracellular ROS and nitrite levels in rat isolated
hepatocytes incubated with or without 0.1% LE and rotenone for 48 h1,²

| | ROS (DCF fluorescence)³ | Nitrite |
|---|---|---|
| Treatment | AU | μmol/106 cells |
| Control | 100 ± 6^{c} | 4.9 ± 0.2^{a} |
| LE | 253 ± 24^{b} | 3.7 ± 0.2^{b} |
| Rotenone | 530 ± 44^{a} | 2.0 ± 0.2^{c} |
| Rotenone + LE | 435 ± 26^{a} | 2.8 ± 0.2^{c} |

2-way ANOVA, P-values

| | | |
|---|---|---|
| LE | P = 0.0005 | P < 0.0001 |
| Rotenone | P < 0.0001 | P < 0.0001 |
| LE × Rotenone | P < 0.0001 | P = 0.0003 |

¹Values are means, SEM, n _ 6. Means in a column without a common superscript letter differ, P < 0.05.
²For ROS measurements, data were log transformed to achieve stabilized variance.
³AU, arbitrary units.

The elevated intracellular ROS levels significantly decreased nitrite levels (Table 4). In cells treated with BSO, nitrite levels and GSH were decreased (Table 5).

TABLE 6
Nitrite and GSH levels in rat isolated hepatocytes
incubated with or without 0.1% LE and BSO for 48 h1,²

| | Nitrite | GSH |
|---|---|---|
| Treatment | μmol/106 cells | nmol/mg protein |
| Control | 5.8 ± 0.4^{a} | 16.7 ± 3.0 |
| LE | 3.4 ± 0.7^{b} | 9.0 ± 1.2 |
| BSO | 2.3 ± 0.3^{c} | 2.2 ± 0.4 |
| BSO + LE | 2.8 ± 0.2^{c} | 1.8 ± 0.2 |

2-way ANOVA, P-values

| | | |
|---|---|---|
| LE | P = 0.0005 | P = 0.012 |
| BSO | P = 0.0001 | P < 0.0001 |
| LE × BSO | P < 0.0001 | NS³ |

¹Values are means, SEM, n = 6. Means in a column without a common superscript letter differ, P < 0.05.
²For GSH measurements, data were log transformed to achieve stabilized variance.
³NS, not significant.

Effects of antioxidants on nitrite levels after exposure to LE.

The inhibitory effect of LE on nitrite levels was attenuated or prevented by various antioxidants (FIG. 3). N-Acetyl-L-cysteine (NAC; 3 mmol/L) increased nitrite levels compared with controls, whereas ascorbate (3 mmol/L) prevented the inhibitory effect of the LE treatment. Resveratrol (600 μmol/L) partially prevented the reduction in nitrite synthesis (FIG. 3).

Hydrophobic antioxidants had no protective effect (data not shown). NAC was able to attenuate the LE-induced reduction of eNOS and iNOS mRNA transcription (FIGS. 2A, 2B), as well as iNOS and eNOS protein levels (FIG. 2C; data not shown). Inhibition of nitrite production was abolished in the presence of NAC.

Effect of LE on TNF stimulated nitrite production.

Cells were preincubated with LE for 48 h and then stimulated with TNF-α for 3-12 h. LE attenuated the TNF-α-stimulative effect of nitrite production at 9 and 12 h after induction (Table 6).

TABLE 6
Nitrite levels of rat isolated hepatocytes incubated with
or without 0.1% LE and TNF-α (100 mU) for 3-12 h¹

| | |
|---|---|
| TNF-α | TNF-α + LE |

| | |
|---|---|
| Time, h | Nitrite (μmol/106 cells) |

| | | |
|---|---|---|
| 3 | 2.1 ± 0.1 | 2.3 ± 0.5 |
| 6 | 2.1 ± 0.2 | 2.5 ± 0.2 |
| 9 | 4.3 ± 0.2 | 3.2 ± 0.1* |
| 12 | 6.5 ± 0.2 | 4.0 ± 0.1* |

¹Values are means SEM, n = 6.
*Different from TNF-α alone, P < 0.05 by t test.

Effect of LE on AP-1 and NF-κB. LE treatment decreased 15% (P <0.05; data not shown) the abundance of the p65 subunit of the of transcription factor NF-□B in the nucleus. LE did not change the abundance of AP1 p-c-Jun subunit in the nucleus (data not shown).

In this study, we investigated the ability of LE to regulate NO production in the liver. An in vitro model of isolated hepatocytes was chosen to emulate hepatic exposure to high levels of circulating TG. Basal levels of NO production might be affected by the isolation procedure because shear stress could induce NO production in this cell model. The results reported here demonstrate that nitrite levels are decreased dose dependently after exposure of primary rat hepatocytes to LE (FIG. 9). Effect of LE on nitrite production and intracellular ROS levels in rat isolated hepatocytes.

It is within the scope of the invention to present a method for treating symptoms of Syndrome X (SX), comprising a step of introducing into a patient a therapeutically effective synergistic amount of an SX -inhibiting cocktail (SIC) comprising polyphenols (P), thiol antioxidants (To) and ascorbic acid (A) wherein the step of introducing reduces intracellular reactive oxygen species (ROS) and/or increases intracellular nitrite (NO), such that reduction of SX is at least 30% greater when compared with reduction of SX provided by administration of each of said P, To and A when separately introduced into patient. It will be understood by a person skilled in the art that the defined reduction of SX by the cocktail as described above, is also provided within the ranges 20% to 25%, 30% to 35%, 40% to 45%, 50% to 55% and above 55%.

It is within the scope of the invention to present a method for treating symptoms of lipotoxicity (L) comprising a step of introducing into a patient a therapeutically effective synergistic amount of a lipotoxicity-inhibiting cocktail (LIC), the LIC comprising polyphenols (P), thiol antioxidants(To) and ascorbic acid (A) such that the said step of introducing reduces ROS and/or increases NO, so that reduction of L is at least 30% greater when compared with said reduction of said L provided by administration of each of said P, To and A when separately introduced into said patient. It will be understood by a person skilled in the art that the defined reduction of L by the cocktail as described above, is also provided within the ranges 20% to 25%, 30% to 35%, 40% to 45%, 50% to 55% and above 55%. [0198] It will further be understood by a person skilled in the art that the aforementioned method can be preferably implemented by introducing into the patient a cocktail wherein the polyphenol is resveratrol.

It will further be understood by a person skilled in the art that the aforementioned method wherein said thiol antioxidant is N-acetyl cysteine.

It is within the scope of the invention to present a method for reducing risk of Arterial, Heart and Related Diseases (AHRD) comprising a step of introducing into a patient a therapeutically effective synergistic amount of an AHRD inhibiting cocktail (AIC) comprising P, To and A. The step of introducing reduces intracellular ROS and/or increases intracellular NO, such that the risk reduction is at least 30% greater when compared with reduction provided by administration of each of said P, To and A when separately introduced into said patient. It will be understood by a person skilled in the art that the defined reduction of L by the cocktail as described above, is also provided within the ranges 20% to 25%, 30% to 35%, 40% to 45%, 50% to 55% and above 55%.

It is within the scope of the invention to present a method for treating arterial and related diseases. These diseases are selected from the group consisting of hypertension, hyperlipidemia, atherosclerosis, arteriosclerosis, coronary artery disease, myocardial infarction, congestive heart failure, stroke, and angina pectoris.

It is within the scope of the invention to present a method for reducing Non-Alcoholic Steatohepatitis (NASH). A therapeutically effective synergistic amount of a NASH-inhibiting cocktail (NIC) comprising P, To and A, is introduced into a patient thereby reducing intracellular reactive oxygen species (ROS) and/or increasing intracellular nitrite (NO). This has the effect of reducing NASH, such that the reduction of said NASH is at least 30% greater when compared with reduction of NASH provided by administration of each of P, To and A when separately introduced into said patient.

It is within the scope of the invention to present a method for treating symptoms of SX, where SX is associated with accumulation of triacylglycerol (TG) in the liver. A synergistic TG accumulation inhibiting cocktail (TIC) is introduced into a patient, having the effect of reducing ROS and/or NO, thereby reducing TG, such that the reduction of TG is at least 30% greater when compared with reduction of TG provided by administration of each of said P, To and A when separately introduced into said patient.

It is within the scope of the invention to present a method for inhibiting lipotoxicity wherein said lipotoxicity is manifested by Free Fatty Acid (FFA) accumulation. This is achieved by introducing into said patient a synergistic FFA accumulation inhibiting cocktail (FIC), wherein the step of introducing reduces ROS and/or increases intracellular nitrite NO, such that the reduction of FFA accumulation is at least 30% greater when compared with reduction of FFA accumulation provided by administration of each of said P, To and A when separately introduced into the patient.

It is within the scope of the invention to implement the above methods by introducing the cocktails into the patient orally.

It is within the scope of the invention to implement the above methods by introducing the cocktails into the patient intravenously.

It is within the scope of the invention wherein a cocktail is presented comprising P, To and A wherein the ratios of the components in the cocktail are such that after introducing the cocktail into a patient, a reduction of SX is at least 30% greater when compared with the reduction of SX provided by administration of each of P, To and A when separately introduced into the patient.

It is within the scope of the invention wherein a cocktail is presented comprising P, To and A wherein the ratios of the components in the cocktail are such that after introducing the cocktail into a patient, a reduction of L is at least 30% greater when compared with the reduction of L provided by administration of each of said P, To and A when separately introduced into patient.

It is within the scope of the invention wherein a cocktail is presented comprising P, To and A wherein the ratios of the components in the cocktail are such that after introducing said cocktail into a patient, a reduction of risk of ARHD is at least 30% greater when compared with said reduction of said risk of AHRD provided by administration of each of said P, To and A when separately introduced into said patient.

It is within the scope of the invention wherein a cocktail is presented comprising P, To and A wherein the ratios of the components in the cocktail are such that after introducing said cocktail into a patient, a reduction of NASH is at least 30% greater when compared with said reduction of NASH provided by administration of each of said P, To and A when separately introduced into said patient.

It is within the scope of the invention wherein a cocktail is presented comprising P, To and A wherein the ratios of the components in the cocktail are such that after introducing said cocktail into a patient, a reduction of TG is at least 30% greater when compared with said reduction of TG provided by administration of each of said P, To and A when separately introduced into said patient.

It is within the scope of the invention wherein a cocktail is presented comprising P, To and A wherein the ratios of the components in the cocktail are such that after introducing said cocktail into a patient, a reduction of FFA is at least 30% greater when compared with said reduction of FFA provided by administration of each of said P, To and A when separately introduced into said patient.

Reference is made to Table 10 below, presenting possible ratios of the components in the cocktail, necessary, according to one embodiment of the invention and in a non-limiting manner for producing synergistic beneficial effects in a patient with respect to the following:

A cocktail comprising synergistic dosages of polyphenols (P), thiol antioxidants (To) and ascorbic acid (A), wherein the ratios of the components in the cocktail are such that after introducing said cocktail into a patient, a reduction of a lipotoxicity-related phenomenon is at least 30% greater than said phenomenon reduction provided by administration of each of said P, To and A, when separately introduced into said patient.

A cocktail useful in treating lipotoxicity-related phenomena and metabolic disorders selected from a group consisting of Syndrome X (SX), Metabolic Syndrome, lipotoxicity (L), Arterial, Heart and Related Diseases (AHRD), Non-Alcoholic Steatohepatitis (NASH), triacylglycerol (TG), diabetes, obesity related disorders, coronary heart disease, Free Fatty Acid (FFA) or any combination thereof

It is within the scope of the invention wherein a cocktail is presented providing reduction of intracellular reactive oxygen species (ROS) and/or increase of intracellular nitrite (NO), such that reduction of SX is at least 30% greater when compared with reduction of SX provided by administration of each of P, T₀ and A when separately introduced into the patient.

It is within the scope of the invention wherein a cocktail is presented providing

reduction of ROS and/or increase of NO, such that reduction of L is at least 30% greater when compared with reduction of L provided by administration of each of P, To and A when separately introduced into the patient.

It is within the scope of the invention wherein a cocktail is presented providing

reduction of ROS and/or increase of NO, such that said risk reduction for AHRD is at least 30% greater when compared with the reduction of risk of AHRD provided by administration of each of P, To and A when separately introduced into the patient.

It is within the scope of the invention wherein a cocktail is presented providing

reduction of ROS and/or increase of NO, such that reduction of NASH is at least 30% greater when compared with the reduction of NASH provided by administration of each of P, To and A when separately introduced into the patient.

It is within the scope of the invention wherein a cocktail is presented providing

reduction of ROS and/or increase of NO, such that reduction of accumulation of TG in the liver is at least 30% greater when compared with the reduction of accumulation of TG in the liver provided by administration of each of P, To and A when separately introduced into the patient.

It is within the scope of the invention wherein a cocktail is presented providing

reduction of ROS and/or increase of NO, such that reduction of FFA accumulation is at least 30% greater when compared with the reduction of FFA accumulation provided by administration of each of P, To and A when separately introduced into the patient.

According to yet another embodiment of the invention, and still in a non-limiting manner, possible ratios are as follows:

TABLE 10
Therapeutic synergistic ratios

| | Ascorbic acid | Resveratrol | N-acetyl cysteine |
|---|---|---|---|
| Ascorbic acid | — | from 1:1000 to | from 1:1000 to |
| | | 1000:1; especially | 1000:1; especially |
| | | from 1:100 to 100:1, | from 1:100 to 100:1, |
| | | e.g., from 1:10 to | e.g., from 1:10 to |
| | | 10:1 | 10:1 |
| N-acetyl cysteine | from 1:1000 to | from 1:1000 to | — |
| | 1000:1; especially | 1000:1; especially | |
| | from 1:100 to 100:1, | from 1:100 to 100:1, | |
| | e.g., from 1:10 to | e.g., from 1:10 to | |
| | 10:1 | 10:1 | |
| Resveratrol | from 1:1000 to | — | from 1:1000 to |
| | 1000:1; especially | | 1000:1; especially |
| | from 1:100 to 100:1; | | from 1:100 to 100:1, |
| | e.g., from 1:10 to | | e.g., from 1:10 to |
| | 10:1 | | 10:1 |

It is acknowledged in this respect that the ratios defined above are molar ratios, or weight ratios. It is however in the scope of the inventions that aforesaid ratios are effective bioactive (e.g., therapeutic) dosage ratios.

It will be noted in the above table that a non limiting example of a polyphenol is provided by resveratrol and a non limiting example of a thiol antioxidant is provided by N-acetyl cysteine.

1. A method for treating symptoms of a lipotoxicity-related phenomenon, comprising at least one step of introducing into a patient a therapeutically effective synergistic amount of a phenomenon-inhibiting cocktail, by said introducing, polyphenols (P), thiol antioxidants (To) and ascorbic acid (A) in synergistic ratios are administered; wherein said introducing of said cocktail reduces inter alia intracellular reactive oxygen species (ROS) and/or increases inter alia intracellular nitrite (NO), such that reduction of said phenomenon is at least 30% greater than said reduction of said phenomenon provided by administration of each of said P, To and A, when separately introduced into said patient.

2. The method according to claim 1, wherein said ratios are effective bioactive dosage ratios ranging from 1:1000 to 1000:1; especially from 1:100 to 100:1; and especially from 1:10 to 10:1.

3. The method according to claim 1, wherein said polyphenol is resveratrol.

4. The method according to claim 1, wherein said thiol antioxidant is N-acetyl cysteine.

5. The method according to claim 1, wherein said lipotoxicity-related phenomenon is a metabolic disorder selected from a group consisting of Syndrome X (SX), Metabolic syndrome, lipotoxicity (L), Arterial, Heart and Related Diseases (AHRD), diabetes, obesity, corner heart disease, Non-Alcoholic Steatohepatitis (NASH), triacylglycerol (TG), Free Fatty Acid (FFA) or any combination thereof.

6. A method for treating arterial and related diseases according to claim 5, wherein said AHRD phenomena are selected from the group consisting of hypertension, hyperlipidemia, atherosclerosis, arteriosclerosis, coronary artery disease, myocardial infarction, congestive heart failure, stroke, and angina pectoris.

7. The method according to claim 1 wherein said introducing is provided orally.

8. The method according to claim 1 wherein said introducing is provided intravenously.

9. A cocktail comprising synergistic dosages of polyphenols (P), thiol antioxidants (To) and ascorbic acid (A), wherein the ratios of the components in the cocktail are such that after introducing said cocktail into a patient, a reduction of a lipotoxicity-related phenomenon is at least 30% greater than said phenomenon reduction provided by administration of each of said P, To and A, when separately introduced into said patient.

10. The cocktail according to claim 9, wherein said ratios are effective bioactive dosage ratios ranging from 1:1000 to 1000:1; especially from 1:100 to 100:1; and especially from 1:10 to 10:1.

11. The cocktail according to claim 9, wherein said lipotoxicity-related phenomenon is a metabolic disorder selected from a group consisting of Syndrome X (SX), Metabolic syndrome, lipotoxicity (L), Arterial, Heart and Related Diseases (AHRD), Non-Alcoholic Steatohepatitis (NASH), triacylglycerol (TG), diabetes, obesity, corner heart disease, Free Fatty Acid (FFA) or any combination thereof.

12. The cocktail according to claim 9, wherein said polyphenol is resveratrol.

13. The cocktail according to claim 9, wherein said thiol antioxidant is N-acetyl cysteine.

14. A food additive comprising the cocktail as defined in claim 9 or in any of its preceding claims.


SEQUENCE LISTING (7 sequences)

SEQ ID NO: 1 - DNA, 20 bp, Artificial
  synthetic
gagcatacccgcacttctgt

SEQ ID NO: 2 - DNA, 20 bp, Artificial
  synthetic
gaagatatctcgggcagcag

SEQ ID NO: 3 - DNA, 20 bp, Artificial
  synthetic
cagcacagagggctcaaagg

SEQ ID NO: 4 - DNA, 19 bp, Artificial
  synthetic
tcgtcggccagctctttct

SEQ ID NO: 5 - protein, 5 aa, Artificial
  synthetic
GAPDH

SEQ ID NO: 6 - DNA, 19 bp, Artificial
  synthetic
tccgccccttccgctgatg

SEQ ID NO: 7 - DNA, 21 bp, Artificial
  synthetic
cacggaaggccatgccagtga